(19) United States
(12) Patent Application Publication
(10) Pub. No.: US 2005/0203633 A1
(43) Pub. Date: Sep. 15, 2005

(54) Lifetime solution for hip dysfunction

(76) Inventors: Alfred Fernandes, Tampa, FL (US); Carol Fernandes, Collierville, TN (US)

Correspondence Address: WRIGHT MEDICAL TECHNOLOGY, INC., 5677 AIRLINE ROAD, ARLINGTON, TN, 38002-9501, US

(21) Appl. No.: 10/791,125

(22) Filed: Mar. 2, 2004

(51) Int. Cl. A61F 2/32

(52) U.S. Cl. 623/22.11

(57) ABSTRACT

A lifetime solution for hip dysfunction is disclosed. Through this solution, by making use of implants already positioned in-situ without the need for removal of the same unless medically indicated, the solution minimizes that amount of implants actually used and the amount of trauma faced by the patient.

### FIELD OF THE INVENTION

The invention primarily concerns the field of hip arthroplasty. However, its principals can be applied to any body joint employing similar mechanics and using implants of similar construction. All patents, printed publications, and articles referred to in this patent application are incorporated by reference herein.

### BACKGROUND OF THE INVENTION

When determining the success rate or viability of a hip arthroplasty regimen, there are multiple considerations, though the considerations are often inter-related. The following are typically the primary considerations:
a) Surgery Longevity;
b) Material Wear;
c) Range Of Movement (ROM); and
d) Pain to the Patient, Incision Size, and Risk of Infection.

To date, as will be described herein, even when taking into account state of the art knowledge in each of these areas, a hip arthroplasty regimen that satisfactorily considers the needs of a young and/or active patient has not been fully developed.

a) Surgery Longevity

It is not atypical for a person undergoing some type of hip surgery to have to undergo another hip surgery later in life. This is especially true in the case of young or active patients. As part of the second surgery, the first surgery may have to be undone, for example by removing a previous implant. This second surgery will often damage and remove an amount of surrounding bone. These second surgeries are often called “revisions”.

During the early stages of the development of hip arthroplasty, the first surgery typically comprised the total primary hip (THR—Total Hip Replacement) and the second surgery comprised the total revision hip. As mentioned in U.S. Pat. No. 4,976,740 once a patient had their THR, the chance that the patient would have to have a second surgery (in the form of a total revision hip) or would have complications increased. This is especially true in younger (less than 40 years old) and more active patients who would outlive the implant and its connection/fixation to the bone. Reconstruction or revision has been generally considered inevitable in total hips as described in U.S. Pat. No. 6,524,843.

In an effort to put off until later in life a patient's THR so as to put off the potential need for additional surgeries, the art of femoral head resurfacing has developed. As discussed, for example, in U.S. Pat. No. 4,976,740, in femoral head resurfacing, the patient's natural acetabulum may or may not be replaced by an acetabular implant when the femoral head resurfacing is done.

b) Wear

Material wear has always been a concern in implant design, especially when plastics were involved. An early solution to this concern was small femoral head diameters to reduce frictional forces between the femoral head and its associated UHMWPE acetabulum. See e.g. USPub 2003/0114935. However, as will be described below, small femoral head diameters lead to limited ROM and increased likelihood of dislocation.

Other solutions to the wear issue were material selection and processing. Highly polished metal-metal finishes were suggested. See U.S. Pat. Nos. 6,120,545 and 6,126,695. Specialized materials, including CoCr treated with Nitrogen gas, see e.g. U.S. Pat. No. 5,308,412, diamond, see, e.g. U.S. Pat. No. 6,626,949, and CrCoMo, see, e.g., U.S. Pat. No. 6,120,545, were also suggested. Ceramics are also now widely used.

In any event, it was thought that early wear would lead to patient disease and the need to replace the implant early in implant selection. Accordingly, it was proposed that very refined surface parameters for preparation of the femoral head should be implemented. See U.S. Pat. No. 6,126,692.

c) ROM

Though as mentioned above, smaller femoral heads resulted in less friction, they also produced less range of motion than a natural hip and more dislocations. See e.g. Bartz, et al., The Effect of Femoral Component Head Size on Posterior Dislocation of the Artificial Hip Joint, Journal of Bone Joint Surgery, 82A: 1300-07 (2000). Early hips had femoral heads on the order of 22-28 mm. Large or “jumbo” femoral heads, having a diameter of at least 32 mm, were known and thought to have superior stability, but thought to wear excessively, and not promoted despite their mechanical stability. Id.

d) Pain to the Patient/Incision Size/Risk of Infection

Obviously, multiple surgeries intensify the pain to the surgery as well as risk of infection and increased wound size. Furthermore, as revision hip components are typically larger than primary or resurfacing hip components, revision incision sizes must typically be larger.

Therefore, there is a need for a lifetime solution in the field of hip surgery that minimizes the amount of surgery imparted on the patient and minimizes the amount of implants used or interchanged.

### SUMMARY OF THE INVENTION

It is an object of the invention to provide a lifetime solution regimen of hip arthroplasty that minimizes pain and risk to the patient over the patient's lifetime.

It is an object of the invention to provide a lifetime solution regimen of hip arthroplasty that is especially suitable to younger patients.

It is an object of the invention to provide a lifetime solution regimen of hip arthroplasty that makes use of implants already positioned in-situ without the need for removal of the same and minimizes that amount of implants actually used.

### BRIEF DESCRIPTION OF THE DRAWINGS

FIGS. 1(a) and 1(b) depict two aspects of a prior art hip arthroplasty system provided by the assignee of this patent application.

FIGS. 2(a), 2(b), 2(c), 2(d) depict various steps in the lifetime solution according to the invention.

FIG. 3 compares a patient's timeline when using a conventional hip arthroplasty regimen with that of the invention.

### DETAILED DESCRIPTION OF THE INVENTION

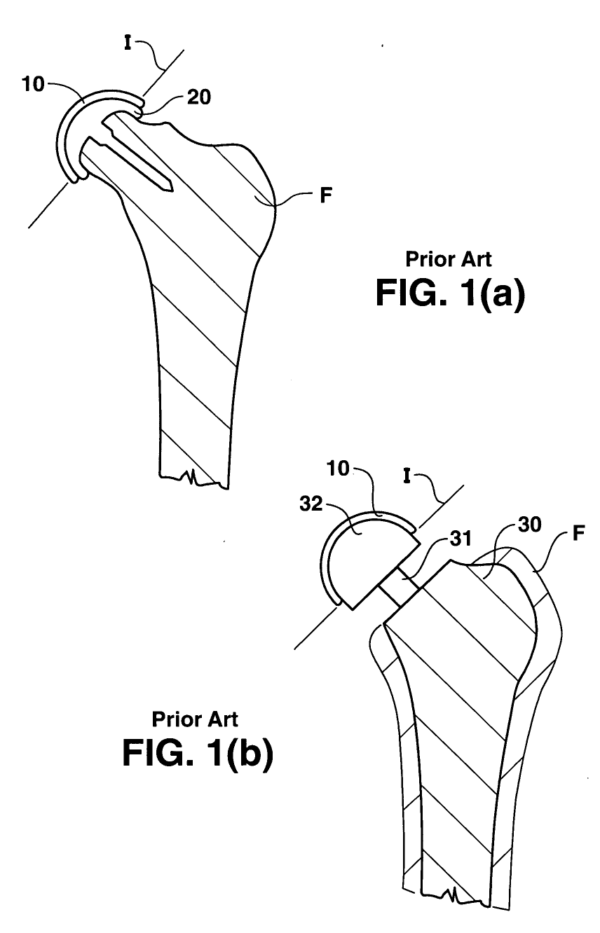

As shown in FIGS. 1(a) and 1(b), Assignee of this patent application has been providing a hip arthroplasty system in which it was possible to go from a total hip resurfacing (FIG. 1(a)) to total primary hip replacement (FIG. 1(b)) re-using the acetabular component from the earlier femoral resurfacing surgery as the acetabular component for the total primary hip. By “re-use”, Applicants mean use as the same acetabular bearing surface in both cases and without being removed between surgeries so as to remain well fixed.

During a total hip resurfacing, as shown in FIG. 1(a), the femoral head of the femur F is replaced with a femoral head resurfacing component 20 and the patient's natural acetabulum of the ilium I is replaced with an acetabular component 10.

During a total primary hip replacement, as shown in FIG. 1(b), a primary hip stem 30 is inserted in the medullary canal of the femur 30. By “primary hip stem”, Applicants also include the associated primary neck 31 and femoral ball/head 32. According to this prior art, the femoral head resurfacing component 20 of FIG. 1(a) is removed and replaced with the hip stem 30 of FIG. 1(b). The diameter of the head of the primary hip stem will typically be the same as that of the head of the femoral head resurfacing component. As previously mentioned, acetabular component 10 is not replaced and remains well fixed.

These particular femoral and acetabular components are generally described in U.S. Pat. No. 6,156,069 and were made of CoCr, highly polished, and of a highly toleranced spherocity (curvature) of at least about 3 microns. It is also possible to use high-strength, low-wear ceramics; polished and toleranced in a similar manner to their CoCr counterparts. Head sizes were considered “jumbo”, i.e., having a head diameter greater than 32 mm to provide stability and greater ROM. However, this size was thought to place the heads in the high friction category.

The acetabular component was a linerless system. In other words, the femoral head will rest directly in the acetabular component and not in an intermediate liner. This allows for the use of larger femoral head sizes.

Conventional wisdom would lead away from re-use of these components because it is typically common practice to always swap out both implants regardless of their quality at the time of surgery due to fears about unseen wear or potential perceived fears about wear. Clinical results and patient follow-up for this system, however, have shown that the concerns shown by those skilled in the art were unwarranted and the same acetabular component has withstood the extended cycle times commensurate with being used as both a hip resurfacing and primary total hip acetabulum without excessive wear or increased metal ions in the blood stream.

Furthermore, these clinical results concerning Assignee's specific products have been generally confirmed in a prestigious study that found that metal-metal hip surfaces do not wear as much as originally thought. Chan et al., Wear and Lubrication of Metal-on-Metal Hip Implants, Clinical Orthopaedics and Related Research, 369:10-24 (1999).

Accordingly, this long term success led Applicant to conclude that despite the conventional wisdom to contrary, it is possible to have individual hip components (e.g., femoral component, acetabular component) capable of extended (5,000,000+) cycle times, including in the metal-metal environment. These extended cycle times would allow for the replacement of only those components that actually need to be replaced due to actual failure or wear and not perceived failure or wear. Furthermore, this would mean if one metallic component is changed, the patient would not necessarily have to be matched up with a new matching component. This would lead to a much more patient friendly lifetime solution for hip arthroplasty.

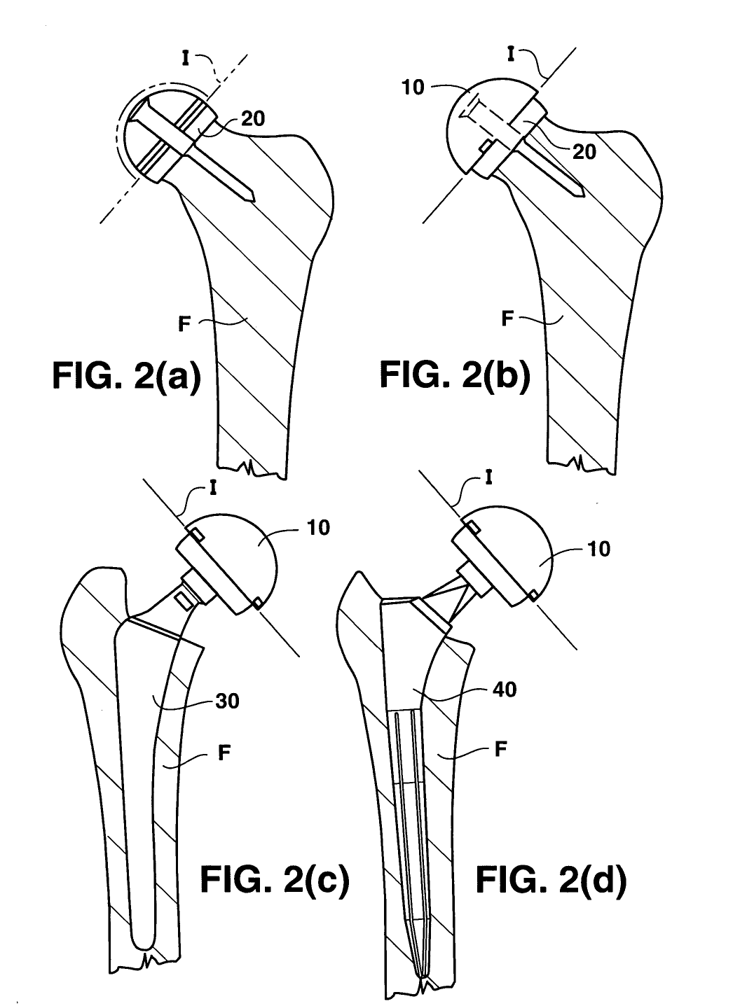

To implement these findings, we have proposed a hip arthroplasty regimen comprising four sequential steps over the life of the average patient, preferably a young or active patient, according to the invention is shown in FIGS. 2(a)-(d).

FIG. 2(a) shows what is generally known as a hemi-resurfacing. During a hemi-resurfacing, the patient's natural femoral head is replaced with a femoral head resurfacing component 20 having a diameter that closely matches the diameter of the patient's natural femoral head (which would be considered to be in the “jumbo” range), but the patient's natural acetabulum is not replaced with an acetabular component.

FIG. 2(b) shows what is generally known as a total resurfacing as has been previously described. During a conventional total resurfacing, the patient's natural femoral head is replaced with a femoral head resurfacing component 20 having a matching diameter as described with respect to a hemi-resurfacing and the patient's natural acetabulum is replaced with an acetabular component 10 implanted in the ilium. The acetabular component will have a matching and tightly toleranced diameter with respect to the femoral head diameter. Typically, for this invention, as previously described, the acetabular component will comprise a linerless system.

FIG. 2(c) shows a total primary hip. During a conventional total hip, the patient's natural femoral head is replaced with a hip stem 30 inserted in the medullary canal of the femur F and having a femoral head and the patient's natural acetabulum is replaced with an acetabular component 10 implanted in the ilium. Furthermore, a total primary hip may comprise replacing a femoral head resurfacing component 20 with a hip stem 30 inserted in the medullary canal of the femur F. Diameter matching is again employed.

FIG. 2(d) shows a total revision hip. During a conventional total revision, a previously implanted femoral component (i.e. hip stem inserted in the medullary canal and having a femoral head) is replaced with a hip stem 40 designed for revision surgery that is inserted in the medullary canal of the femur F and having a femoral head and the patient's natural acetabulum is replaced with an acetabular component implanted in the ilium. Finally, as above, diameter matching is again employed.

Bone loss (primarily in the femur F) is minimal in a femoral resurfacing and increases from total hip to revision hip and with each revision.

According to the invention, during the later surgeries of FIGS. 2(b)-(d), unless there is some surgical indication, the acetabular component 10 implanted in the patient during the original femoral head resurfacing surgery is not changed. This invention is primarily concerned with the average patient that does not require special revision acetabulum due to severe bone damage or loss in the ilium, etc. However, of course, if at some point in the patient's life the patient does need such a revision acetabulum, the conventional acetabulum implanted during the total resurfacing step (FIG. 2(b)) can be replaced with any number of styles of revision acetabulums.

Accordingly, while each of the steps of FIGS. 2(a)-(b) have been known in the art alone, the invention comprises the re-use of implanted femoral and acetabular hip implant components within the patient as the patient moves from one step to another, rather than the replacement of implanted components as the patient moves from one step to the next. As mentioned above, by “re-use”, Applicant's mean use of the same implanted component from one step to the next and without being removed between surgeries so as to remain well fixed.

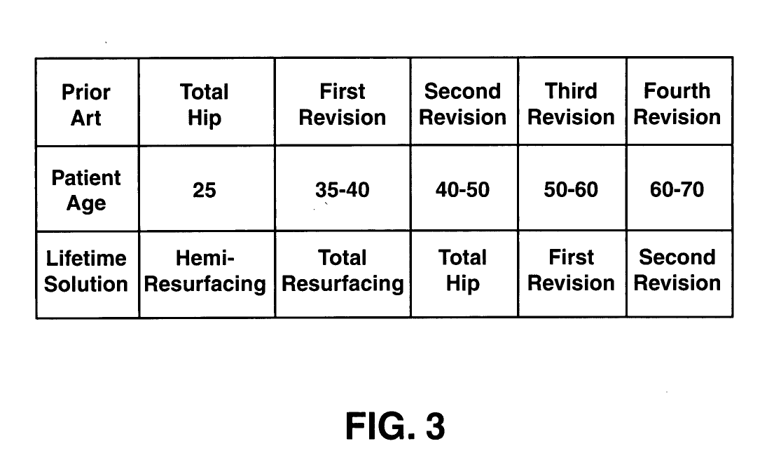

FIG. 3 depicts how implementation of the invention would improve the life of a patient with a hip problem. FIG. 3 compares the approximate ages at which the patient would undergo the various procedures of the prior art THA regimen and compares them against when the patient would undergo those procedures proposed in the lifetime solutions regimen of the invention.

For an exemplary 25 year old patient, with only a femoral head problem, e.g., avascular necrosis or osteoporosis of the femoral neck, a typical starting point, if the patient undergoes a prior art THR, the patient can expect undergoing their first total revision surgery at around age 35-45 and then once every approximately 15 years. Each revision surgery will become more complicated, remove more bone, place the patient at greater risk and result in more pain.

According to the invention, for the same patient, if the patient would be first given, at age 25, as is generally known, a femoral head resurfacing only (i.e., natural acetabulum left alone), less risky total femoral head resurfacing (i.e., implanting an acetabular component in the patient while leaving in the femoral head resurfacing component) and total primary hips can precede the first revision hip, which does not occur until approximately age 60. At this point, the patient will have lost significantly less bone undergoing the inventive sequence.

Of course, even if the patient must go from the hemi-resurfacing directly to a total primary or start at a total resurfacing, the lifetime solutions idea of reusing, for example, the acetabular component, still provides for increased benefits to the patient. Indeed, all surgeries will be easier because it is now known that the acetabular implant does not have to be changed from surgery to surgery.

It is true that the prior art already contemplates other regimens in which a total hip is not the first step in the patient's lifetime hip solution. Indeed, attempts at internal femoral head repair such as by forming a cavity in the femoral head, see, e.g., U.S. Pat. Nos. 5,928,239 and 6,358,251, seek to even put off femoral head hemi-resurfacing. Furthermore, a method where a liner is placed over the implanted acetabular component has been proposed. See USPub 2003/0181987. However, the degree to which these methods: (1) work in young and active patients and (2) set up an overall progression between various steps of a lifetime solution leading ultimately to a total revision hip much later in life are not defined.

1. A method of using a femoral head resurfacing component, comprising:
(a) implanting said femoral head resurfacing component on the femoral neck of the patient during a first surgery to be matched with the natural acetabulum of the patient; and
(b) leaving said femoral head resurfacing component in said body of the patient during a second surgery in which an acetabular component to be matched with the femoral head resurfacing component is implanted in the patient.

2. The method according to claim 1, wherein the femoral head is jumbo.

3. The method according to claim 1, wherein the femoral component is metallic or ceramic.

4. The method according to claim 1, wherein the acetabular component is metallic or ceramic.

5. The method according to claim 1, including the step of removing said femoral head resurfacing component and replacing it with a primary hip stem while leaving said acetabular component in said body.

6. The method according to claim 1, wherein the acetabular component is linerless.

7. A method of performing hip arthroplasty over the life of a patient, comprising using an acetabular component that is capable of being used as both a primary and then the revision acetabular component.

8. The method of claim 7, wherein said acetabular component is also capable of being used with a femoral head resurfacing component.

9. A method of performing hip arthroplasty over the life of a patient, comprising using the same acetabular component as both the patient's primary and then revision acetabular component.

10. The method according to claim 9, wherein said method further includes using said same acetabular component during the patient's femoral head resurfacing prior to said primary hip.

11. A method of performing hip arthroplasty over the life of a patient, comprising:
(a) implanting a femoral resurfacing component in the patient during a first surgery to be matched with the patient's natural acetabulum;
(b) implanting an acetabular component to be matched with said femoral resurfacing component during a second surgery;
(c) replacing said femoral resurfacing component with a primary stem component having a femoral ball during a third surgery without replacing said acetabular component.

12. The method according to claim 11, further comprising replacing said primary stem with a revision stem and a new femoral ball during a fourth surgery without replacing said acetabular component.

13. The method according to claim 11, further comprising providing said acetabular component with predetermined surface characteristics.

14. The method according to claim 13, wherein said step of providing said acetabular component with predetermined surface characteristics further comprises providing said acetabular component with highly toleranced spherocity and smoothness.

15. A method of using a hip stem in a patient in which an acetabular implant has been implanted on a prior occasion, including the step of implanting said hip stem without replacing said acetabular implant.
